(19) United States
(12) Patent Application Publication
(10) Pub. No.: US 2016/0045649 A1
(43) Pub. Date: Feb. 18, 2016

(54) Surgical Storage Assembly

(71) Applicant: Debra J. Post, Edison, NJ (US)

(72) Inventor: Debra J. Post, Edison, NJ (US)

(21) Appl. No.: 14/458,670

(22) Filed: Aug. 13, 2014

(51) Int. Cl. A61M 1/00

(52) U.S. Cl. CPC A61M 1/008; A61M 2209/088

(57) ABSTRACT

A surgical storage assembly for preventing a plurality of surgical drains from becoming impeded includes a belt that may be placed around a user. A plurality of pockets is coupled to the belt. A plurality of surgical drains is removably positioned within an associated one of the plurality of pockets.

### BACKGROUND OF THE DISCLOSURE

### Field of the Disclosure

The disclosure relates to storage devices and more particularly pertains to a new storage device for preventing a plurality of surgical drains from becoming impeded.

### SUMMARY OF THE DISCLOSURE

An embodiment of the disclosure meets the needs presented above by generally comprising a belt that may be placed around a user. A plurality of pockets is coupled to the belt. A plurality of surgical drains is removably positioned within an associated one of the plurality of pockets.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a surgical storage assembly according to an embodiment of the disclosure.

FIG. 2 is a left side view of an embodiment of the disclosure.

FIG. 3 is a front view of an embodiment of the disclosure.

FIG. 4 is a back view of an embodiment of the disclosure.

FIG. 5 is a top view of an embodiment of the disclosure.

FIG. 6 is an in-use view of an embodiment of the disclosure.

FIG. 7 is a schematic view of a method of utilizing an embodiment of the disclosure.

### DESCRIPTION OF THE PREFERRED EMBODIMENT

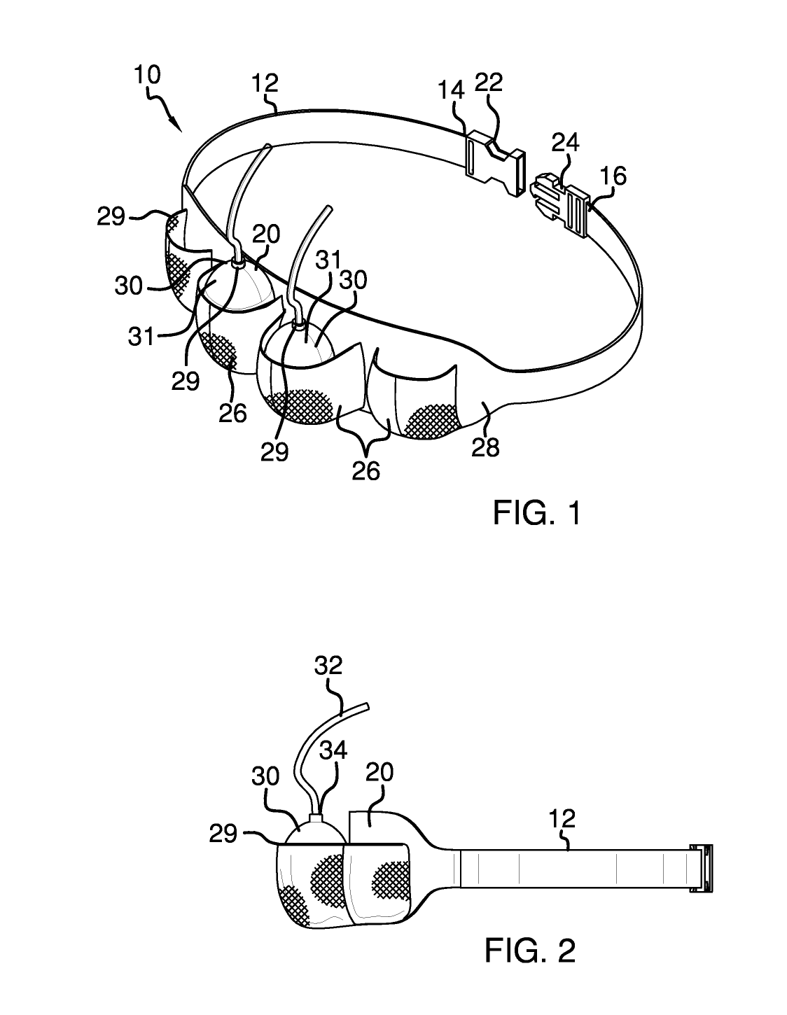

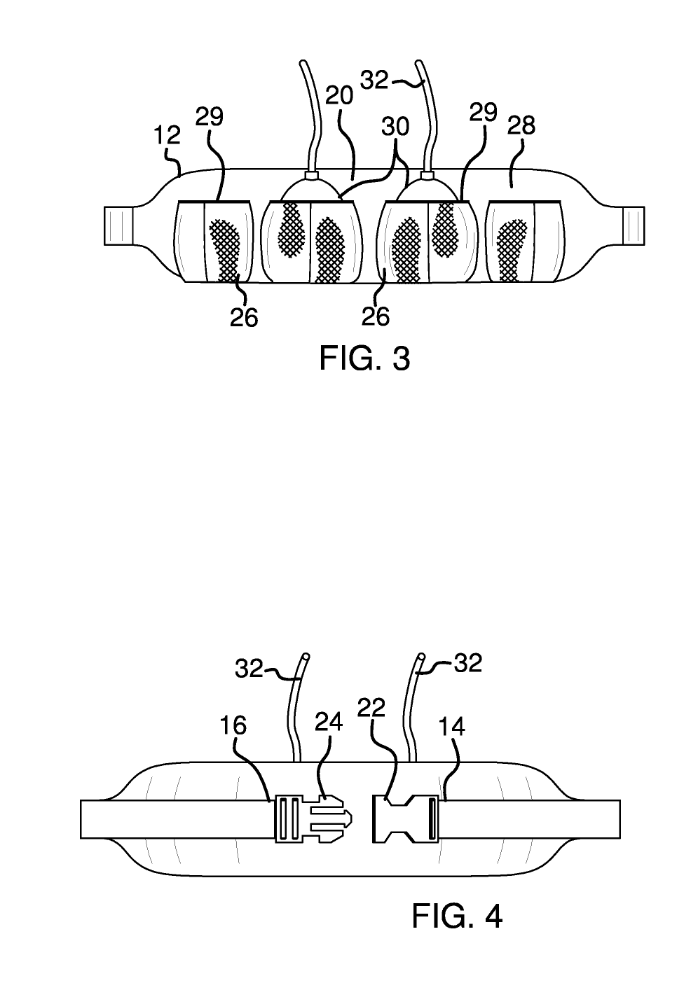

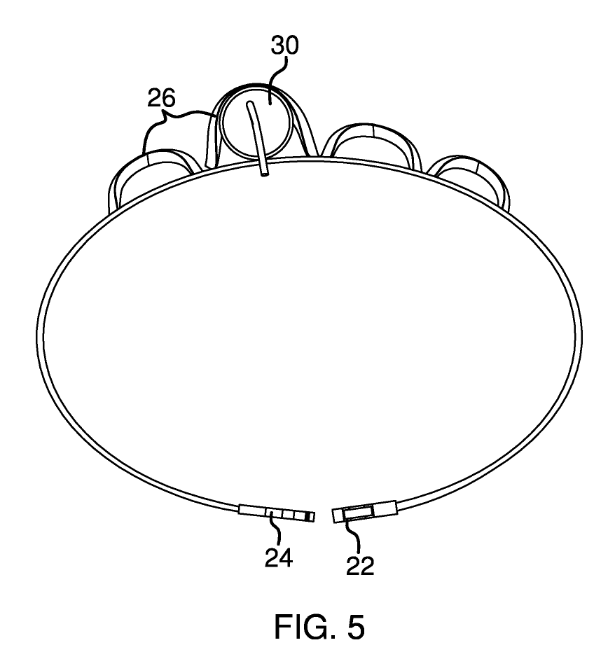

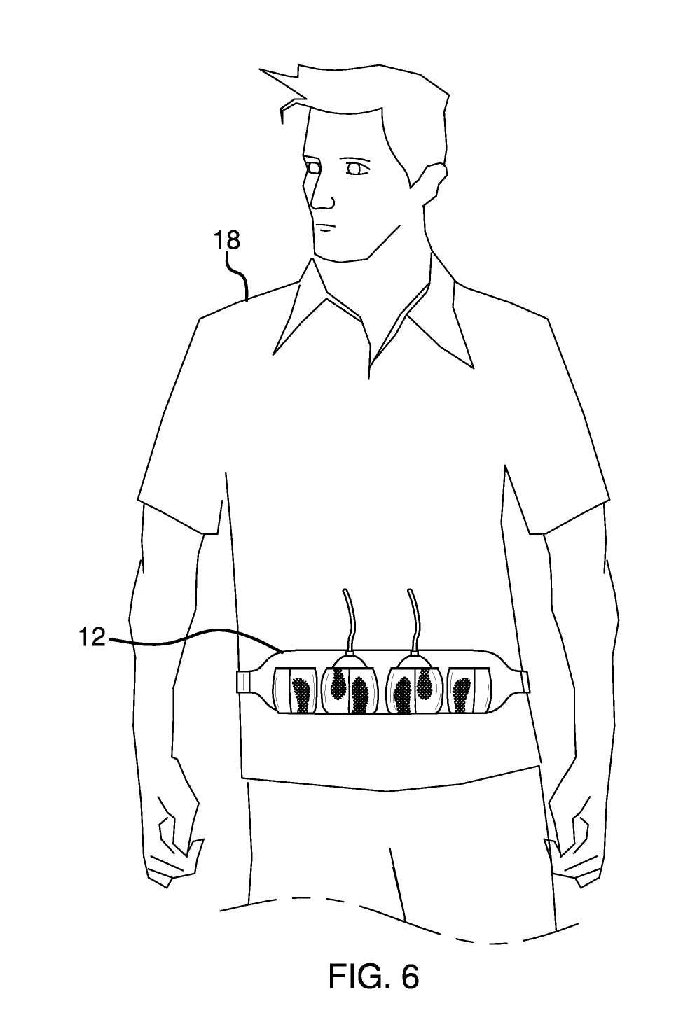

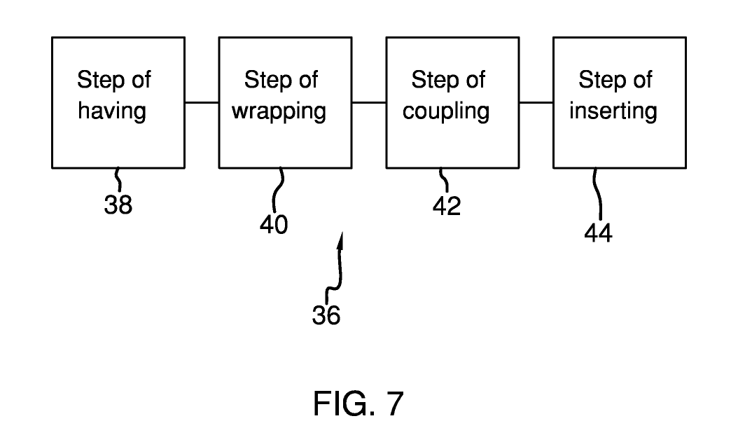

With reference now to the drawings, and in particular to FIGS. 1 through 7 thereof, a new storage device embodying the principles and concepts of an embodiment of the disclosure and generally designated by the reference numeral 10 will be described.

As best illustrated in FIGS. 1 through 7, the surgical storage assembly 10 generally comprises a belt 12. The belt 12 has a first end 14 and a second end 16. The belt 12 may be placed around a user 18. A middle 20 of the belt 12 has a height that is greater than each of the first 14 and second 16 ends of the belt 12.

A first buckle 22 is coupled to the first end 14 of the belt 12. Additionally, a second buckle 24 is coupled to the second end 16 of the belt 12. The second buckle 24 engages the first buckle 22 after the belt 12 is placed around the user 18. Moreover, the belt 12 is retained on the user 18. The user 18 may be a surgical patient.

A plurality of pockets 26 is provided. Each of the pockets 26 is coupled to a front side 28 of the belt 12. The pockets 26 are evenly spaced apart and laterally distributed along the middle 20 of the belt 12. The pockets 26 may each be comprised of a resiliently stretchable material.

A plurality of surgical drains 30 removably positioned within an associated one of the plurality of pockets 26. The plurality of surgical drains 30 each has an outer wall 31. The outer wall 31 of each of the surgical drains 30 is curvilinear. The surgical drains 30 each has a spherical shape. Moreover, the surgical drains 30 is each substantially hollow.

A top 29 of each of the pockets 26 is open. The top 29 of each of the pockets 26 may insertably receive an associated one of a plurality of surgical drains 30. The plurality of surgical drains 30 may each be a surgical bulb of any conventional design. Additionally, a hose 32 is coupled to an upper end 34 of each of the surgical drains 30. The pockets 26 each retains the associated one of the surgical drains 30 so the surgical drains 30 are prevented from becoming entangled or otherwise impeded.

In use, a method 36 of retaining the plurality of surgical drains 30 on the user 18 includes a step 38 of having the belt 12, the first 22 and second 24 buckles each coupled to the belt 12, the plurality of pockets 26 coupled to the belt 12 and the plurality of surgical drains 30. The method 36 also includes a step 40 of wrapping the belt 12 around the user 18. Continuing, the method 36 includes a step 42 of coupling the first 22 and second 24 buckles together. The method 36 finally includes a step 44 of inserting the plurality of surgical drains 30 into an associated one of the plurality of pockets 26.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A surgical storage assembly comprising:
a belt configured to be placed around a user;
a plurality of pockets coupled to said belt; and
a plurality of surgical drains removably positioned within an associated one of said plurality of pockets.

2. The surgical storage assembly according to claim 1, further comprising said belt having a first end and a second end.

3. The surgical storage assembly according to claim 2, further comprising a first buckle coupled to said first end of said belt.

4. The surgical storage assembly according to claim 3, further comprising a second buckle coupled to said second end of said belt, said second buckle engaging said first buckle after said belt is placed around the user such that said belt is retained on the user.

5. The surgical storage assembly according to claim 1, further comprising said pockets each being coupled to a front side of said belt.

6. The surgical storage assembly according to claim 5, further comprising said pockets being evenly spaced apart and laterally distributed along a middle of said belt.

7. The surgical storage assembly according to claim 6, further comprising a top of each of said pockets being open such that said top of each of said pockets insertably receives an associated one of said plurality of surgical drains

8. The surgical storage assembly according to claim 7, further comprising said pockets each retaining the associated one of said surgical drains such that said pockets prevents said plurality of surgical drains from becoming entangled or otherwise impeded.

9. A surgical storage assembly comprising:
a belt having a first end and a second end, said belt being configured to be placed around a user;
a first buckle coupled to said first end of said belt;
a second buckle coupled to said second end of said belt, said second buckle engaging said first buckle after said belt is placed around the user such that said belt is retained on the user;
a plurality of pockets each being coupled to a front side of said belt, said pockets being evenly spaced apart and laterally distributed along a middle of said belt;
a plurality of surgical drains configured to be positioned within an associated one of said plurality of pockets;
a top of each of said pockets being open such that said top of each of said pockets insertably receives an associated one of said plurality of surgical drains; and
said pockets each retaining the associated one of said surgical drains such that said pockets prevents said plurality of surgical drains from becoming entangled or otherwise impeded.

10. A method of retaining a plurality of surgical drains on a user, the steps of the method comprising:
having a belt, a first buckle and a second buckle each coupled to said belt, a plurality of pockets coupled to said belt and a plurality of surgical drains;
wrapping said belt around the user;
coupling said first and second buckles together; and
inserting the plurality of surgical drains into an associated one of said plurality of pockets.
